(19) United States
(12) Patent Application Publication
(10) Pub. No.: US 2025/0095642 A1
(43) Pub. Date: Mar. 20, 2025

(54) NATURAL LANGUAGE BASED CLINICAL RECOMMENDATION SYSTEM

(71) Applicant: GE Precision Healthcare LLC, Waukesha, WI (US)

(72) Inventors: Soumya Ghose, Niskayuna, NY (US); Sanand Sasidharan, Bengaluru (IN); Sanghee Cho, Niskayuna, NY (US); Akshit Achara, Bengaluru (IN); Rakesh Mullick, Bengaluru (IN); Anuradha Kanamarlapudi, Bengaluru (IN); Fiona Ginty, Niskayuna, NY (US); Annamraju Ravi Bhardwaj, Bengaluru (IN); Sundararajan Mani, Brookfield, WI (US); Brion Sarachan, Troy, NY (US)

(21) Appl. No.: 18/468,593

(22) Filed: Sep. 15, 2023

(51) Int. Cl. G10L 15/197; G06N 3/0455; G06N 3/09; G10L 15/06; G10L 15/16; G10L 15/22; G16H 10/60; G16H 50/20; G16H 70/20

(52) U.S. Cl. CPC G10L 15/197; G06N 3/0455; G06N 3/09; G10L 15/063; G10L 15/16; G10L 15/22; G16H 10/60; G16H 50/20; G16H 70/20

(57) ABSTRACT

The current disclosure provides methods for an automated clinical recommendation system that generates clinical recommendations for patients of a health care system based on natural language queries submitted by care providers of the health care system. The natural language queries may be typed into a user interface (UI) of the clinical recommendation system, or submitted by voice via a microphone. The clinical recommendation system provides a clinically explainable disease state of a patient based on patient data included in a query, and recommends a next course of action (e.g., a treatment) based on clinical guidelines and population statistics, in a manner that reduces a current burden of clinicians in consulting digital clinical manuals via a series of time-consuming and cumbersome interactions with a graphical user interface (GUI) of the digital clinical manuals.

### TECHNICAL FIELD

Embodiments of the subject matter disclosed herein relate generally to automated clinical recommendation systems for patient care, and more specifically, a clinical recommendation system that relies on a natural language interface.

### BACKGROUND

Health care providers may rely on clinical guidelines when making patient treatment decisions. The clinical guidelines may be digital clinical guidelines available for consultation by the health care providers online via a computer system and/or network of a health care system. A care provider may search for relevant sections in the clinical guidelines, and navigate through information presented in the clinical guidelines using control elements of a graphical user interface (GUI). The care provider may perform a sequence of actions to retrieve or display desired treatment advice, and wait for output to be displayed before selecting a next action. For example, the care provider may have to answer a series of prompts, or make relevant selections in a sequence of dialog boxes (e.g., a wizard). The care provider may enter various patient data into online forms using a keyboard. As a result of having to perform such actions, interacting with online clinical guidelines may be cumbersome and/or time consuming, and may reduce an amount of time care providers spend interacting with patients.

### SUMMARY

In one example, the current disclosure addresses the issues described above with method for a clinical recommendation system, comprising generating an automated response to a natural language query received from a care provider in real time using a first, large language model (LLM) of the clinical recommendation system, the LLM trained on a first set of training data generated by applying a second, recommendation model to anonymized patient data, the recommendation model based on clinical guidelines; verifying the automated response outputted by the LLM using a third, prediction model; and in response to the automated response being verified, displaying the automated response on a display device of the clinical recommendation system.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows a block schematic diagram of an automated clinical recommendation system, in accordance with one or more embodiments of the present disclosure;

FIG. 2 shows a high-level block schematic diagram illustrating a workflow for training a clinical recommendation system, in accordance with one or more embodiments of the present disclosure;

FIG. 3A shows a block schematic diagram illustrating a workflow for training a large language model (LLM) and a reward model of the clinical recommendation system using supervised learning, in accordance with one or more embodiments of the present disclosure;

FIG. 3B shows a block schematic diagram illustrating a workflow for training the LLM using reinforcement learning, in accordance with one or more embodiments of the present disclosure;

FIG. 4 shows a block schematic diagram illustrating a workflow for using the clinical recommendation system to provide guidance to a care provider, in accordance with one or more embodiments of the present disclosure;

FIG. 5 is a flowchart showing an exemplary high-level method for training the clinical recommendation system, in accordance with one or more embodiments of the present disclosure;

FIG. 6 is a flowchart showing an exemplary high-level method for optimizing a performance of an LLM of the clinical recommendation system, in accordance with one or more embodiments of the present disclosure;

FIG. 7 is a flowchart showing an exemplary high-level method for providing a clinical recommendation to a care provider using the clinical recommendation system, in accordance with one or more embodiments of the present disclosure;

FIG. 8 is an exemplary portion of a recommendation model of the clinical recommendation system, in accordance with one or more embodiments of the present disclosure; and

FIG. 9 shows an exemplary prediction model of the clinical recommendation system, in accordance with one or more embodiments of the present disclosure.

The drawings illustrate specific aspects of the described systems and methods. Together with the following description, the drawings demonstrate and explain the structures, methods, and principles described herein. In the drawings, the size of components may be exaggerated or otherwise modified for clarity. Well-known structures, materials, or operations are not shown or described in detail to avoid obscuring aspects of the described components, systems and methods.

### DETAILED DESCRIPTION

An automated clinical recommendation system is provided herein, which may generate clinical recommendations for patients of a health care system based on natural language queries submitted to the automated clinical recommendation system by care providers of the health care system. The natural language queries may be typed by the care providers into a user interface (UI) of the clinical recommendation system, or submitted by voice, for example, via a microphone. In this way, the proposed clinical recommendation system may allow clinicians to interact with clinical guidelines in a more natural manner, by requesting appropriate treatment guidance in natural language. The clinical recommendation system may provide a clinically explainable disease state of a patient and recommend a next course of action (e.g., a treatment) based on clinical guidelines and population statistics, in a manner that reduces a current burden of clinicians in consulting digital clinical manuals via a series of time-consuming and cumbersome interactions with a graphical user interface (GUI) of the digital clinical manuals. A natural language-based interaction would reduce the digital device interactions of clinicians, thereby allowing the clinicians to devote more time to interacting with patients. Additionally, the proposed clinical guidance system would offer transparency to patients on a care pathway of a disease and provide patients with better context of a clinical procedure.

The automated clinical recommendation system may rely on various models to generate the clinical recommendations, including at least a natural language processing (NLP) model for answering queries posed as questions; a recommendation model for predicting an appropriate treatment for a patient of the health care system, which may be used to train the NLP model; and a prediction model for verifying an output of the NLP model. The various models may include AI models, such as a machine learning (ML) or deep learning (DL) models, generative AI or a different kind of model. For example, one or more of the various models may be implemented as a convolutional neural network (CNN) model including a plurality of hidden layers. The models may include classification or prediction models, probabilistic models (e.g., Bayesian models), statistical models, or a different type of model.

The recommendation model may be based on clinical guidelines used by the health care system, depending on a type of patient and/or pathology presented. For example, the National Comprehensive Cancer Network (NCCN) Clinical Practice Guidelines in Oncology (NCCN Guidelines®) may be a first set of clinical guidelines used for cancer patients. Additional sets of clinical guidelines may be used for specific types of cancer. For example, for patients with prostate cancer, the Prostrate Imaging Reporting & Data System (PI-RADS®) may be used as a second set of clinical guidelines. In various embodiments, the recommendation model may be implemented as a decision tree or knowledge graph that may be traversed to determine a suggested treatment based on patient data.

The NLP model may be a private implementation of a commercial, open source, or in-house large language model (LLM), such as OpenAI's GPT, which may be trained to process the natural language queries and provide responses based on the recommendation model. The LLM may be trained using a combination of supervised learning, with ground truth data generated manually based on the recommendation model, and reinforcement learning, where the LLM is optimized using a reward model. After the LLM is trained and optimized, the clinical recommendation system may receive a natural language query from a care provider (e.g., a question about a course of action regarding a patient), and submit the natural language query to the LLM as a prompt. The LLM may output a response to the prompt, which may include the suggested treatment.

The output of the LLM may be verified by the prediction model. The prediction model may be trained on training data generated by applying the recommendation model to historical patient data. In one embodiment, the prediction model takes patient data included in a natural language query to the LLM and a recommended treatment included in a natural language response to the query outputted by the LLM as input data, and outputs a probability of the recommended treatment being the most appropriate treatment for the patient, based on the historical patient data.

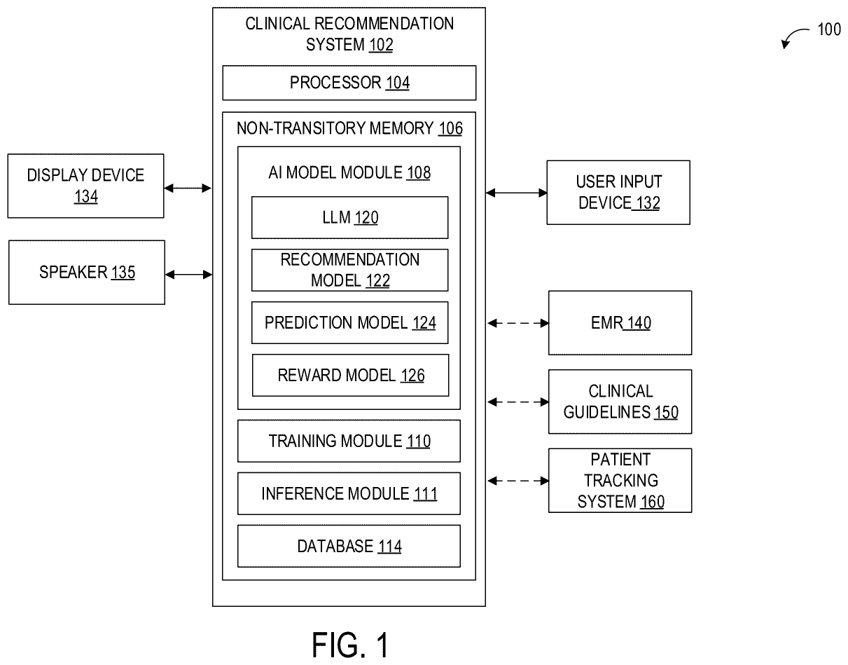

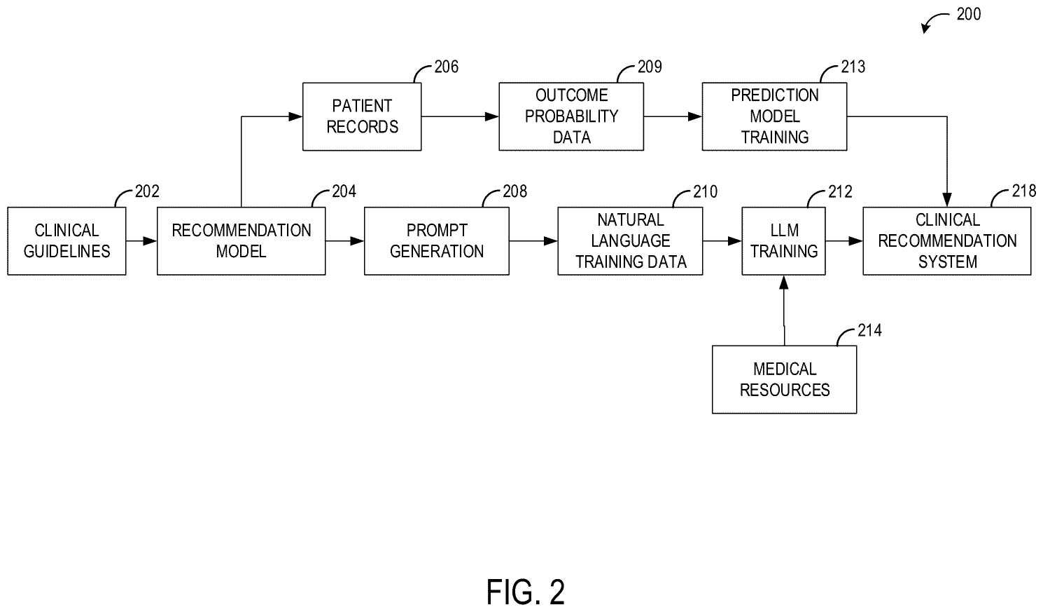

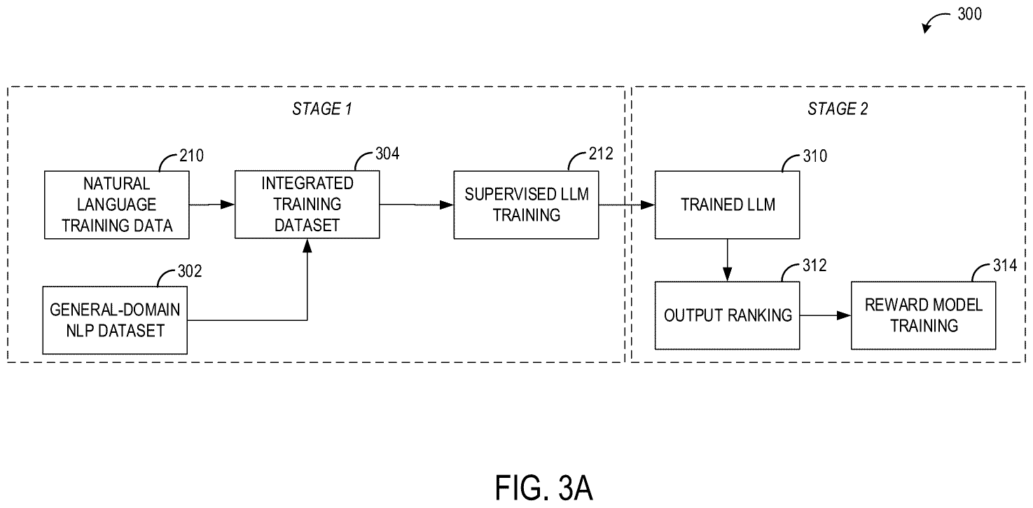

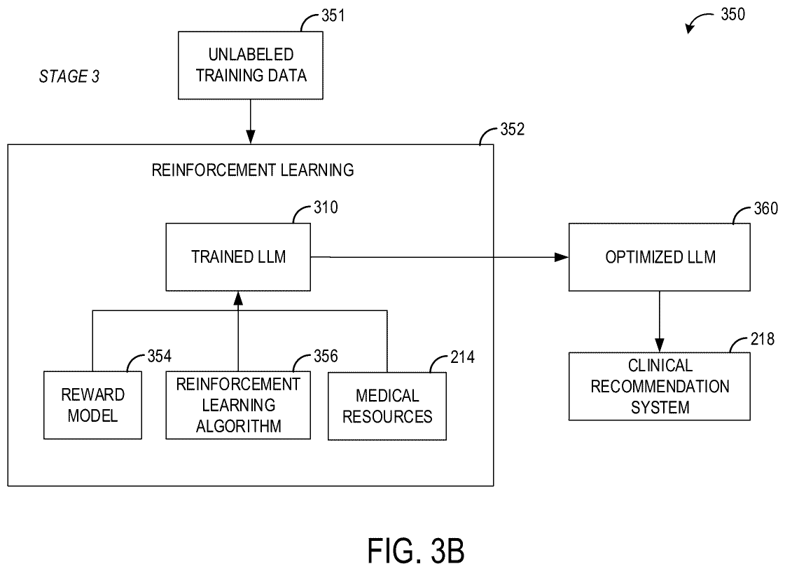

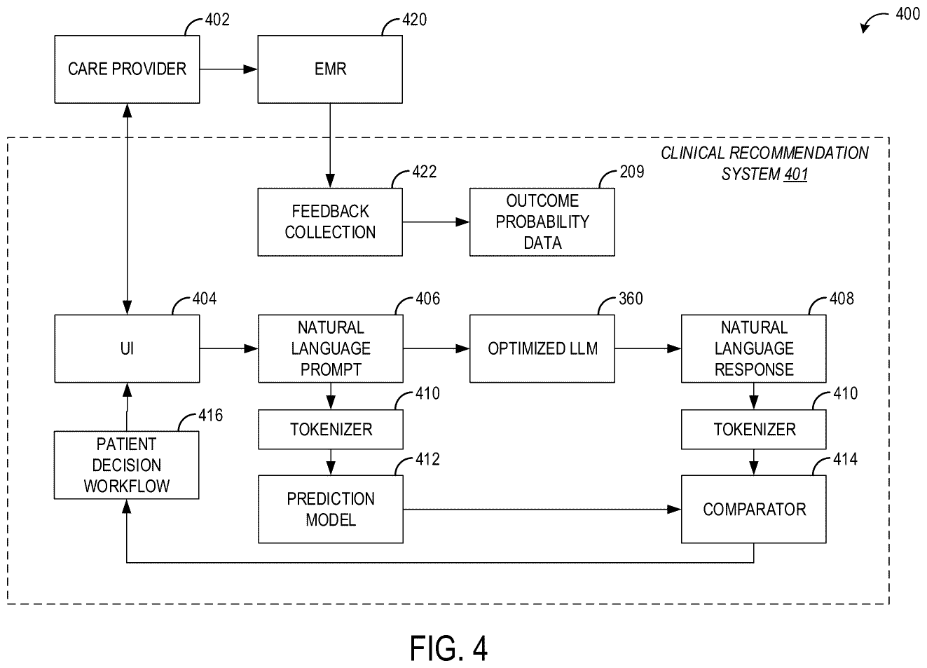

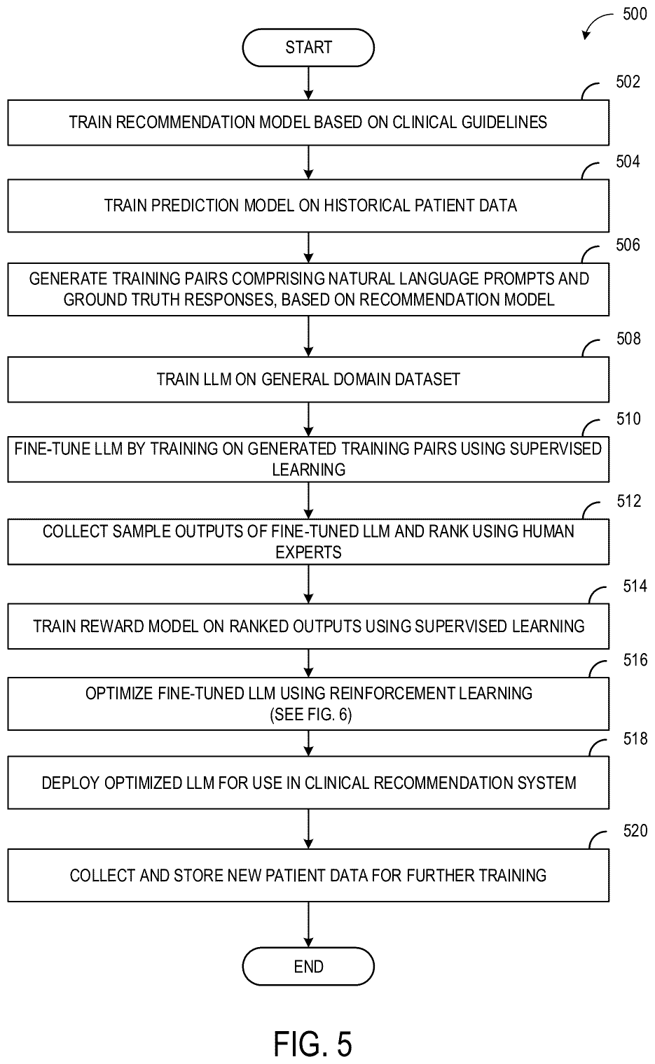

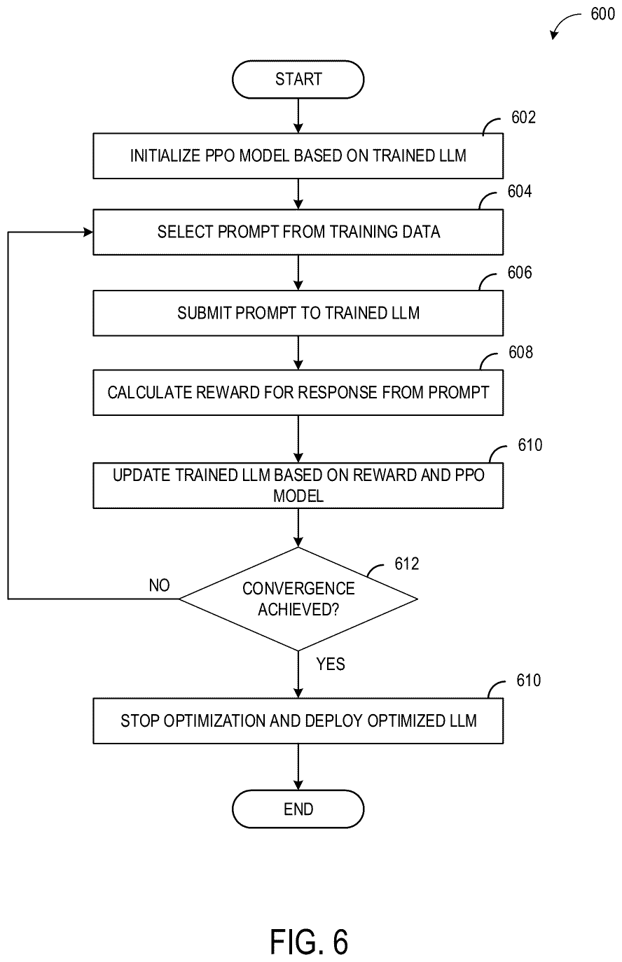

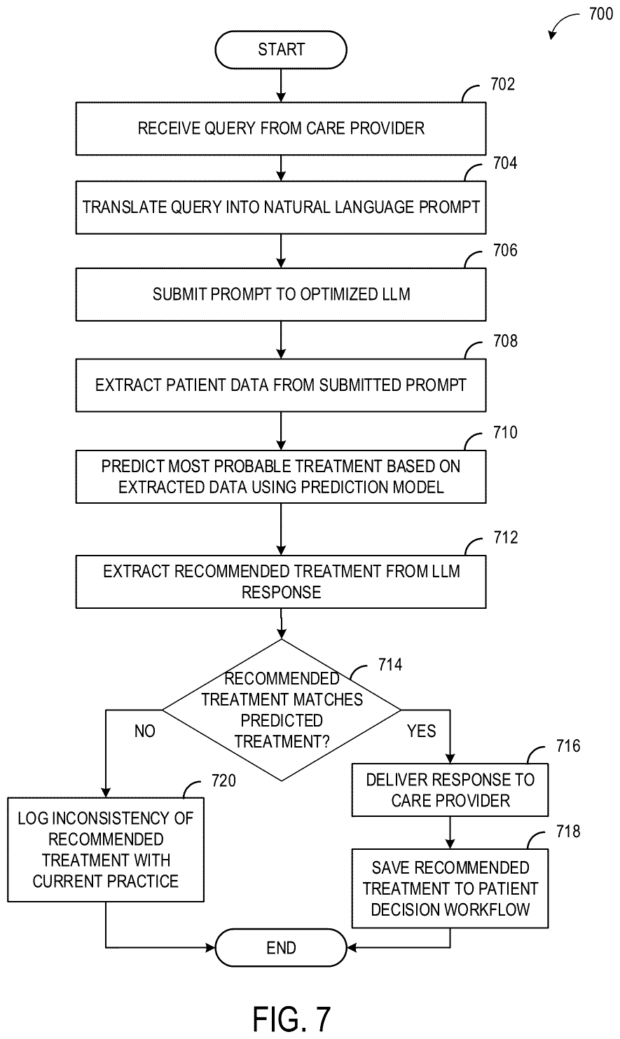

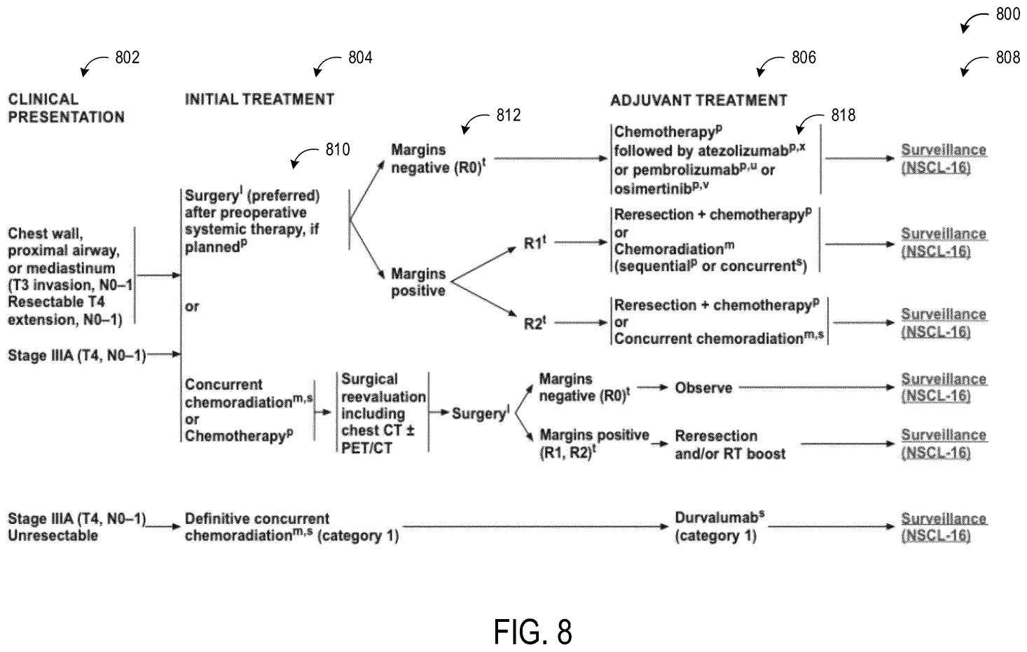

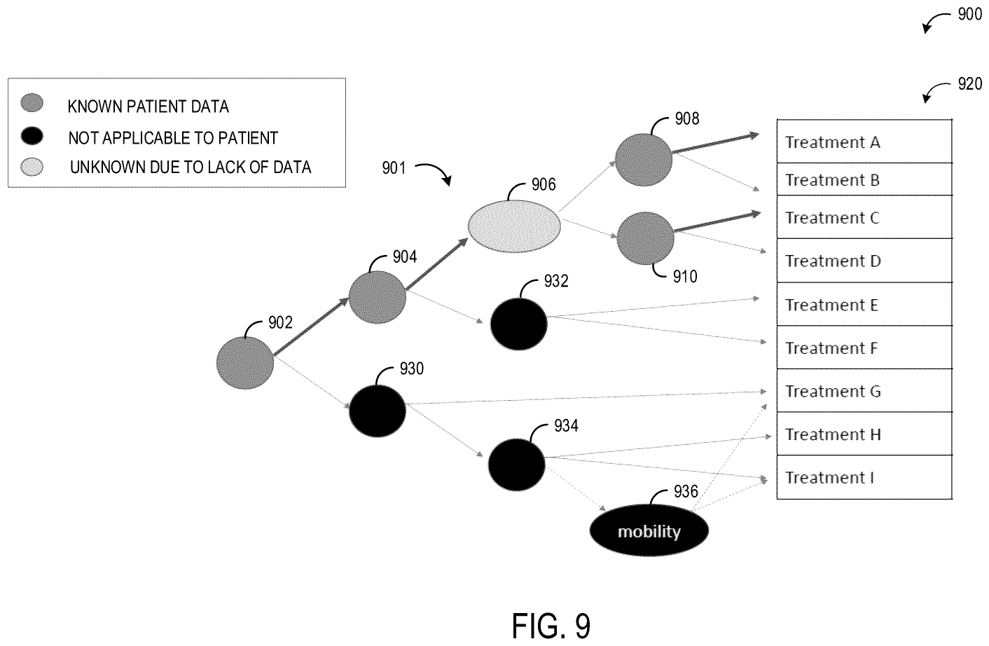

An exemplary clinical recommendation system is shown in FIG. 1. The clinical recommendation system may rely on an LLM, which may be trained in accordance with the workflow shown in FIG. 2. Specifically, the LLM may be trained using a combination of supervised learning, as shown in FIG. 3A, and reinforcement learning, as shown in FIG. 3B. The trained LLM may be used to generate treatment recommendations for patients in accordance with the workflow shown in FIG. 4. The LLM may be trained by following one or more steps of the method shown in FIG. 5. Training the LLM may include optimizing a performance of the LLM by following one or more steps of the method shown in FIG. 6. The clinical recommendation system may be used to generate responses to natural language queries submitted by a care provider by following one or more steps of the method shown in FIG. 7. An exemplary recommendation model used to generate training data for the LLM is shown in FIG. 8. A prediction model may be used to verify outputs of the LLM, such as the prediction model shown in FIG. 9.

FIG. 1 shows an exemplary automated clinical recommendation system 102, in accordance with an embodiment. Clinical recommendation system 102 may provide treatment guidance to health care providers, based on historical patient data and reference materials including clinical guidelines. In some embodiments, at least a portion of clinical recommendation system 102 is disposed at a device (e.g., workstation, edge device, server, etc.) communicably coupled to one or more healthcare and/or hospital networks and computer systems via wired and/or wireless connections, and can receive or access medical data (including patient data) stored in the one or more healthcare and/or hospital computer systems. The medical data may include online clinical reference materials, such as a set of digital clinical guidelines 150, which may be used by care providers to provide treatment alternatives to patients. Clinical recommendation system 102 may also be operably/communicatively coupled to a user input device 132, a display device 134, and/or a speaker 135. In some examples, user input device 132 may be a shared input device of the one or more healthcare and/or hospital computer systems, and display device 134 may be a shared input device of the one or more healthcare and/or hospital computer systems.

Clinical recommendation system 102 includes a processor 104 configured to execute machine readable instructions stored in non-transitory memory 106. Processor 104 may be single core or multi-core, and the programs executed thereon may be configured for parallel or distributed processing. In some embodiments, processor 104 may optionally include individual components that are distributed throughout two or more devices, which may be remotely located and/or configured for coordinated processing. In some embodiments, one or more aspects of processor 104 may be virtualized and executed by remotely-accessible networked computing devices configured in a cloud computing configuration.

Non-transitory memory 106 may store an AI model module 108, a training module 110, an inference module 111, and a database 114. AI model module 108 may include various AI models, such as ML and/or DL models. In particular, AI model module may include an LLM 120 that may be trained to respond to natural language queries regarding treatment alternatives posed by health care providers, as described in greater detail below in reference to FIG. 5. The AI module may include a reward model 126, which may be used to train the LLM using reinforcement learning, as described in greater detail below in reference to FIG. 6. The AI module may also include a recommendation model 122, which may be used to recommend a treatment option based on clinical guidelines 150 as described in greater detail below in reference to FIG. 7. The AI module may also include a prediction model 124, which may be used to verify an output of the LLM. In various embodiments, the one or more AI models may include neural network models such as convolutional neural networks (CNNs), generative adversarial networks (GAN), transformers and traditional classifiers. In some embodiments, the one or more AI models may include a Bayesian network, such as the CNN depicted in FIG. 9. AI model module 108 may include trained and/or untrained neural networks and may further include various data, or metadata pertaining to the one or more AI models stored therein.

Training module 110 may comprise instructions for training the one or more AI models stored in AI model module 108 and/or other types of AI, ML, DL, statistical, or other models. Training module 110 may include instructions that, when executed by processor 104, cause clinical recommendation system 102 to conduct one or more of the steps of method 500 of FIG. 5 for training LLM 120. In some embodiments, training module 110 may include instructions for implementing one or more gradient descent algorithms, applying one or more loss functions, and/or training routines, for use in adjusting parameters of one or more neural networks of AI model module 108. Training module 110 may include training datasets for the one or more AI models of AI model module 108.

Inference module 111 may include instructions for deploying one or more trained AI models, for example, to respond to natural language queries submitted to clinical recommendation system 102 by a care provider. In particular, inference module 111 may include instructions that, when executed by processor 104, cause clinical recommendation system 102 to conduct one or more of the steps of method 700 of FIG. 7.

Database 114 may include anonymized historical patient data, which may be retrieved and compiled from an electronic medical record (EMR) 140 to be used in one or more training sets for training the one or more AI models of AI model module 108. The historical patient data may include a variety of data from patients of the health care system regarding pathologies suffered by the patients, including treatments performed on the patients and outcomes of the treatments. In some embodiments, database 114 may include patient decision workflows of one or more patients, where a patient decision workflow is a stored history of clinical decisions made with respect to a relevant patient using clinical recommendation system 102. The patient decision workflows are described in greater detail below in reference to FIG. 4.

User input device 132 may comprise one or more of a touchscreen, a keyboard, a mouse, a trackpad, a microphone, a motion sensing camera, or other device configured to enable a user to interact with clinical recommendation system 102. In one example, user input device 132 may enable a user to submit a question regarding a patient to clinical recommendation system 102 in natural language. For example, the user (e.g., a care provider) may type the question into clinical recommendation system 102 using a keyboard, or speak the question into a microphone.

Display device 134 may include one or more display devices utilizing virtually any type of technology. In some embodiments, display device 134 may comprise a computer monitor. Display device 134 may be combined with processor 104, non-transitory memory 106, and/or user input device 132 in a shared enclosure, or may be peripheral display devices and may comprise a monitor, touchscreen, projector, or other display device known in the art, which may enable a user to view responses to queries submitted to clinical recommendation system 102, and/or interact with various data stored in non-transitory memory 106.

It should be understood that clinical recommendation system 102 shown in FIG. 1 is for illustration, not for limitation. Another appropriate clinical recommendation system may include more, fewer, or different components.

FIG. 2 shows a schematic diagram of a first workflow 200 for training an LLM (e.g., LLM 120) to respond to natural language queries submitted to a clinical recommendation system 218, which may be a non-limiting example of clinical recommendation system 102 of FIG. 1. A method for training the LLM is described in reference to FIG. 5, which may follow first workflow 200. The steps indicated in first workflow 200 may be performed based on instructions stored in a non-transitory memory of clinical recommendation system 218, such as in training module 110 of non-transitory memory 106 of clinical recommendation system 102 of FIG. 1.

First workflow 200 starts with the creation of a recommendation model 204 for recommending one or more treatment options for a patient to a care provider using the clinical recommendation system. Recommendation model 204 may be generated from and/or based on one or more sets of clinical guidelines 202 (e.g., clinical guidelines 150 of FIG. 1). Clinical guidelines 202 may be digital guidelines available online and/or on a computer system or network of a healthcare system using the clinical recommendation system. Clinical guidelines 202 may include different reference guidelines for different types of patients and/or pathologies. For example, a first set of clinical guidelines may relate to patients who have or are suspected of having cancer; a second set of clinical guidelines may relate to patients who have or are suspected of having an auto-immune disease; a third set of clinical guidelines may relate to patients who are suffering from traumatic wounds; and so on. In one example, clinical guidelines 202 includes the NCCN Guidelines®.

In some examples, a plurality of recommendation models 204 may be created for a corresponding plurality of clinical guidelines 202. For example, a first recommendation model 204 may be created for patients having cancer; a second recommendation model 204 may be created for patients with auto-immune diseases; a third recommendation model 204 may be created for patients suffering from traumatic wounds; and so on. Each recommendation model 204 may be used to determine a most appropriate treatment for a patient based on a corresponding set of clinical guidelines 202.

In various embodiments, recommendation model 204 is a decision tree. The decision tree may be generated manually from a corresponding set of clinical guidelines by human experts. An exemplary decision tree is shown in FIG. 8. In other embodiments, recommendation model 204 may be a different type of model. For example, in one embodiment, recommendation model 204 may be a knowledge graph.

Recommendation model 204 may be used for an LLM training step 212, and a prediction model training step 213. LLM training step 212 may rely on a first training data set, and prediction model training step 213 may rely on a second training data set, where the first training data set and the second training data set may both be generated with the aid of recommendation model 204. For LLM training step 212, a natural language training data set 210 may be generated manually during a prompt generation stage 208 by human experts, who generate realistic natural language prompts and associated responses recommending treatments using recommendation model 204 as a guide. The LLM may be trained on data pairs including the realistic natural language prompts, where the associated responses are included as ground truth data.

In some embodiments, retrieval augmented generation (RAG) may be used during the training of the LLM, where the natural language prompts submitted to the LLM may include instructions to consult one or more medical resources 214 to generate an appropriate response. The instructions may provide additional information relevant to a prompt. A sequence-to-sequence RAG model may take a sequence of inputs, and generate a corresponding sequence of outputs that could be fine-tuned for specific applications like clinical guidelines. For example, an input to the RAG model may include queries such as “Given stage 4 lung cancer with tumor, node, and metastasis (TNM) status [xxx] and surgical margin [yyy], what should be the next steps?”. Fine-tuned RAG would use inputs of the RAG model to retrieve a set of relevant documents, including clinical trials and journal articles, which would then be concatenated to provide relevant outputs/prompts for the fine-tuned LLMs. By using RAG when submitting the prompt, a performance of the LLM and an accuracy of a result returned by the LLM may be increased.

For prediction model training step 213, outcome probability data 209 may be generated by applying recommendation model 204 to a set of anonymized historical patient records 206. In other words, historical patient records may be analyzed to determine a set of probabilities that treatments recommended by recommendation model 204 have a highest probability of success. The set of probabilities may then be used to train the prediction model to predict a treatment for a given patient scenario with a highest probability of success. The prediction model may be used to verify an output of the LLM on the same patient scenario, as described in greater detail below. After the prediction model and the LLM have been trained, the trained prediction model and the trained LLM may be incorporated into clinical recommendation system 218.

Training of the LLM is described in greater detail in relation to the workflow diagrams of FIGS. 3A and 3B. In particular, training of the LLM may be accomplished in three stages. During a first stage of the training, supervised learning is used to train the LLM on the human-generated training data. During a second stage of the training, a reward model is trained using supervised learning, based on ground truth data generated by manual ranking of LLM outputs by human experts. During a third stage of the training, the LLM is optimized using reinforcement learning, based on the trained reward model.

Turning now to FIG. 3A, a second workflow 300 is depicted including the first and second stages of LLM training described above. The steps indicated in second workflow 300 may be performed based on instructions stored in a training module of clinical recommendation system 218, such as in training module 110 of FIG. 1. For the supervised LLM training 212 of FIG. 2, a training dataset is generated from two sources: the natural language training data 210 of FIG. 2, and a general-domain NLP dataset 302. General-domain NLP dataset 302 may include natural language training data pairs of prompts and responses obtained under commercial data usage agreement. General-domain NLP dataset 302 may be used to train the LLM to respond to queries regarding specific treatment plans for patients presenting with different pathologies. To train the LLM to provide clinical treatment recommendations, an integrated training dataset 304 may be compiled including both natural language training data 210 created using the recommendation model 204 of FIG. 2, and general-domain NLP dataset 302. Both natural language training data 210 and general-domain NLP dataset 302 may be used concurrently in a supervised setting to fine-tune the LLM.

After training on the integrated training dataset 304, an output of a trained LLM 310 may be used to train a reward model for further optimization of the trained LLM 310 using reinforcement learning. Outputs of trained LLM 310 may be sampled and ranked by human experts in an output ranking step 312. The ranked outputs may be used to train the reward model during a reward model training step 314. During reward model training step 314, the reward model may be trained using supervised learning, on training pairs including an output of the trained LLM 310 and a corresponding ranking as ground truth data.

FIG. 3B shows the third workflow 350 corresponding to a third stage of the training process, where trained LLM 310 is optimized during a reinforcement learning step 352. The steps indicated in third workflow 350 may be performed based on instructions stored in the training module of clinical recommendation system 218. During reinforcement learning step 352, the trained LLM 310 is trained on various corpora of unlabeled training data 351, using a reward model 354 trained at reward model training step 314 of FIG. 3A and a reinforcement learning algorithm 356. In various embodiments, reinforcement learning algorithm 356 may be the Proximal Policy Optimization (PPO) reinforcement learning algorithm. Optimization of a trained LLM is described in greater detail below in reference to FIG. 6. Once trained LLM 310 has been optimized at reinforcement learning step 352, an optimized LLM 360 may be deployed to clinical recommendation system 218 for use in a clinical environment during an inference stage.

FIG. 4 shows a fourth workflow 400 for using optimized LLM 360 to provide clinical treatment recommendations to a care provider 402 of a health care system via a clinical recommendation system 401, which may be a non-limiting example of clinical recommendation system 218 of FIGS. 2-3B and/or clinical recommendation system 102 of FIG. 1. The steps indicated in fourth workflow 400 may be performed based on instructions stored in an inference module of clinical recommendation system 401, such as inference module 111 of FIG. 1.

In accordance with fourth workflow 400, care provider 402 may submit a question to clinical recommendation system 401 via a user interface (UI) 404 of clinical recommendation system 401. For example, UI 404 may include a graphical user interface (GUI) displayed on display device 134 of clinical recommendation system 102 of FIG. 1, and care provider 402 may interact with UI 404 via user input device 132 (e.g., a keyboard, mouse, microphone, etc.). UI 404 may include a microphone, where the question may be submitted as an audio file recorded via the microphone. For example, in one embodiment, a GUI of UI 404 may be displayed on a smart phone of care provider 402, and care provider 402 may submit the question via a microphone of the smart phone. The question may be regarding a patient. For example, care provider 402 may seek a recommendation from clinical recommendation system 401 about one or more courses of action and/or treatment options to take with the patient.

Clinical recommendation system 401 may receive the question via UI 404, and may convert the question into a natural language prompt 406. Natural language prompt 406 may be generated from decision trees as observed in clinical guidelines and/or knowledge graphs of a specific disease to incorporate relevant domain knowledge. Natural language prompt 406 may be submitted to optimized LLM 360 of FIG. 3B. Optimized LLM 360 may output a natural language response 408, which may answer the question of care provider 402. However, prior to displaying natural language response 408 to care provider via UI 404, natural language response 408 may be verified using a separate prediction model 412. Prediction model 412 may be trained at prediction model training step 213 described in reference to FIG. 2.

To verify natural language response 408, a tokenizer 410 may extract relevant tokens from natural language prompt 406, where the relevant tokens include patient data describing a state of the patient, which optimized LLM 360 relies on to generate natural language response 408. The patient data describing the state of the patient may also correspond to elements of recommendation model 204. For example, in an embodiment where recommendation model 204 is a decision tree, the patient data may be used to traverse the decision tree to determine a treatment option recommended by the clinical guidelines (e.g., clinical guidelines 150 of FIG. 1 and/or clinical guidelines 202 of FIG. 2).

The patient data extracted from natural language prompt 406 by tokenizer 410 may be inputted into prediction model 412. Prediction model 412 may output a prediction of a treatment option for the patient with a highest probability of being successful, based on the patient data and the outcome probability data 209 generated based on historical patient data as described in reference to FIG. 2, on which prediction model 412 was trained. Similarly, tokenizer 410 may be used to extract relevant tokens from natural language response 408, where the relevant tokens include a specific treatment option recommended by optimized LLM 360.

A comparator 414 may then determine whether the specific treatment option recommended by optimized LLM 360 matches the treatment option with the highest probability of success outputted by prediction model 412. If the treatment option recommended by optimized LLM 360 matches the treatment option with the highest probability of success outputted by prediction model 412, natural language response 408 may be added to a patient decision workflow 416 and delivered to care provider 402 via UI 404. For example, natural language response 408 may be displayed on a display device of the clinical recommendation system (e.g., display device 134), or natural language response 408 may be converted to an audio signal and played to care provider 402 via a speaker (e.g., speaker 135) or device including a speaker, such as a smart phone.

Patient decision workflow 416 may be a stored representation of a plurality of clinical decisions recommended and/or made using clinical recommendation system 401, which may be used as a curated history of patient care received by the patient. For example, patient decision workflow 416 may show a progression of the patient through various decision points at which clinical decisions were recommended. Patient decision workflow 416 may be stored in a database of the clinical recommendation system, such as database 114 of clinical recommendation system 102 of FIG. 1. In some embodiments, patient decision workflow 416 may include additional information about the clinical recommendations, such as, for example, information on which the clinical recommendations were based, other options that were not selected, etc. For example, the information on which the clinical recommendations were based may be extracted from natural language prompt 406, the other options that were not selected may be extracted from a recommendation model of clinical recommendation system 401 (e.g., recommendation model 204 of FIG. 2), and so on.

As an example of how the patient decision workflow might be used, optimized LLM 360 may recommend a first treatment at a first decision point in a patient journey of the patient. A new patient decision workflow 416 may be generated including patient data included in a first natural language prompt 406, and the first recommended treatment. The patient decision workflow 416 may be stored in a database of the clinical recommendation system. Care provider 402 may treat the patient with the first recommended treatment. When the first recommended treatment is performed on the patient, care provider 402 may update the patient decision workflow 416 to indicate that the first recommended treatment was followed. At a second decision point of the patient journey, care provider 402 may submit a second natural language prompt 406 including new patient data, and requesting a second treatment recommendation. Optimized LLM 360 may generate a second natural language response 408 with the second treatment recommendation. The second treatment recommendation may be included (e.g., added) to the patient decision workflow 416. Care provider 402 may treat the patient with the second recommended treatment. When the second recommended treatment is performed on the patient, care provider 402 may update the patient decision workflow 416 to indicate that the second recommended treatment was followed. In this way, a record of the recommendations made at each decision point of the patient journey and the decisions made in response to the recommendations may be included in the patient decision workflow 416.

Further, in some embodiments, patient decision workflow 416 may used to guide treatment of a new patient with a similar profile, diagnosis, etc. as the patient. For example, when submitting natural language prompt 406, care provider 402 may request one or more patient decision workflows 416 of patients having a similar diagnosis. Optimized LLM 360 may retrieve the one or more patient decision workflows 416 and display them in UI 404. Care provider 401 may review and select a most similar patient decision workflow 416, and customize the most similar patient decision workflow 416 for the new patient. Alternatively, in some examples, care provider 401 may include instructions in natural language prompt 406 for optimized LLM 360 to select and customize the most similar patient decision workflow 416 for the new patient. By customizing a stored patient decision workflow 416 of a prior patient, a recommended clinical decision may be determined more quickly and with less use of resources of clinical recommendation system 401 than by generating the recommended clinical decision as described above.

Additionally or alternatively, one or more patient decision workflows 416 may be incorporated into the recommendation model or used in conjunction with the recommendation model to recommend a treatment to a patient. For example, the recommendation model may be a Bayesian network, where probabilities incorporated into the Bayesian network may be adjusted based on the one or more patient decision workflows 416.

Further still, in some embodiments, one or more patient decision workflows 416 may be used to verify a performance of clinical recommendation system 401 and/or track errors in a performance of the LLM at various stages of patient care. For example, a decision recommended using optimized LLM 360 may be compared to similar decisions made based on similar information in one or more patient decision workflows 416. If the recommended decision is different from the similar decisions, the recommended decision may not be provided to care provider 402.

If the treatment option recommended by optimized LLM 360 does not match the treatment option with the highest probability of success outputted by prediction model 412, natural language response 408 may not be delivered to care provider 402 via UI 404, and an indication of an inconsistency between the proposed treatment and the clinical guidelines may be recorded by clinical recommendation system 401. The inconsistency may be used to refine optimized LLM 360 in further training to more closely adhere to the clinical guidelines. In other examples, the inconsistency may be used to adjust the clinical guidelines.

Care provider 402 may follow the course of action recommended by clinical recommendation system 401, and apply the treatment outputted by optimized LLM 360. As the treatment is applied to the patient, patient data may be updated in an EMR 420 of the healthcare system in accordance with a patient journey of the patient. The patient data may include a final outcome of the treatment. In various embodiments, the updated patient data including the final outcome may be retrieved by clinical recommendation system 401 during a feedback collection step 422. The updated patient data may be saved in a training database of clinical recommendation system 401 (e.g., database 114). The updated patient data including the final outcome may be incorporated into the outcome probability data 209 used to retrain and increase a performance of prediction model 412. The updated patient data may also be incorporated into the natural language training data 210 used to retrain and further optimize optimized LLM 360. In this way, a performance of optimized LLM 360, prediction model 412, and clinical recommendation system 401 may be continuously increased over time.

Referring now to FIG. 5, a method 500 is shown for training a LLM to recommend treatment options for a patient to a care provider of a health care system, via a clinical recommendation system such as clinical recommendation system 102 of FIG. 1. The LLM may be a non-limiting example of LLM 120. Method 500 may be performed by a processor of the clinical recommendation system (e.g., processor 104), based on instructions stored in a non-transitory memory of the clinical recommendation system (e.g., AI model module 108 and training module 110).

Method 500 begins at 502, where the method includes training a recommendation model based on one or more sets of clinical guidelines. Training the recommendation model may include mapping elements of the one or more sets of clinical guidelines to a hierarchical structure for modeling decisions, such as a decision tree. The hierarchical structure may then be traversed based on patient data, until reaching a decision point at which one or more courses of action may be recommended, based on the options available at the decision point. The decision tree/knowledge graph/recommendation model may be created in a rule-based framework from existing clinical guidelines, literature, domain knowledge of a disease and/or using routinely collected clinical training data to create a graph or tree based machine learning models in a supervised, semi-supervised, self-supervised or un-supervised machine learning framework. An example recommendation model implemented as a decision tree is shown in FIG. 8.

Referring to FIG. 8, a portion of a recommendation model 800 is shown, where recommendation model 800 is a decision tree. The portion of recommendation model 800 comprises a framework for making treatment decisions in regard to a theoretical patient, where decision points of the decision tree are depicted in columns reading from left to right. In the depicted portion, one or more exemplary clinical presentations of a patient is shown in a first column 802. One or more initial recommended treatments for patients presenting with a clinical presentation are shown in a second column 804. One or more recommended adjuvant treatments corresponding to different results of the initial recommended treatments is shown in a third column 806. A final course of action following the initial and adjuvant treatments may be to monitor the patient for signs of improvement, as depicted in a fourth column 808.

As an example of how recommendation model 800 may be used, a patient may present to a care provider with stage III lung cancer. In accordance with recommendation model 800, two alternative initial treatments are recommended at first column 802. At a first decision point 810, surgery may be recommended as an initial treatment, or concurrent chemoradiation/chemotherapy may be recommended as an initial treatment. If surgery is selected at first decision point 810, different adjuvant treatment options are recommended at a second decision point 812, depending on whether margins are positive or negative. If the margins are negative at decision point 812, an adjuvant treatment 818 is recommended, comprising chemotherapy followed by treatment with one or more medications. If the margins are positive at decision point 812, different adjuvant treatments are recommended, depending on additional criteria. If a different initial treatment is selected, the decision tree may be traversed accordingly until reaching a different decision point recommending one or more adjuvant treatments. In this way, recommendation model 800 may be traversed based on clinical data obtained for a patient until a decision point is reached, which may indicate a subsequent course of action based on the clinical data.

As described below, recommendation model 800 may be used to generate a first set of training data for training an LLM of the clinical recommendation system, and/or a second set of training data for training a prediction model of the clinical recommendation system.

Returning to FIG. 5, at 504, method 500 includes training a prediction model on historical patient data. The prediction model may be trained to receive (new) patient data as input, and output a predicted treatment with a highest probability of being selected by care providers, based on the historical patient data. The training data for the prediction model may include training pairs comprising patient data indicating a state of the patient, such as a state of a disease of the patient, and patient outcome data, meaning treatments applied to the patient based on the state.

In some embodiments, the prediction model may include a Bayesian network that is trained to learn a set of conditional probabilities of the treatment being selected based on historical treatment data. The Bayesian network may provide a probabilistic expectation of an outcome on a patient care pathway, based on population statistics and current and prior clinico-pathological variables. In other embodiments, the prediction model may be a CNN trained to output the recommended treatment with the highest probability of being selected based on the historical patient data. In still other embodiments, a different type of model or neural network may be used.

The prediction model may allow for tracking deviations from usual clinical practice based on the population statistics. In other words, the Bayesian network could be used to identify an unusual patient journey in the care pathway, and highlight decision points where the deviation occurred. Deviation tracking may be used to update the clinical guidelines, and/or to realign clinical practice with the clinical guidelines. For example, the recommendation model may be updated or expanded to include more or different decision points. If clinical data is found to be inconsistent with the clinical guidelines, a new decision point may be added.

Referring briefly to FIG. 9, an exemplary Bayesian network 900 is shown, where Bayesian network 900 is an implementation of a prediction model used to predict a treatment recommended for a patient within a clinical recommendation system. Bayesian network 900 may have a hierarchical structure that follows or is based on a hierarchical structure of the recommendation model. As such, Bayesian network 900 includes a plurality of nodes 901, where each node of nodes 901 may correspond to a decision point of the recommendation model (e.g., recommendation model 800 of FIG. 8). Each node 901 links to different subsequent options for patient treatment, where the nodes 901 may be traversed to determine a recommended treatment for the patient, similar to the recommendation model. A predicted treatment may be indicated at an output layer 920 of Bayesian network 900. For a given patient, some of nodes may be based on known patient data, such as nodes 902, 904, 908, and 910, while other nodes may not be applicable to the patient, such as nodes 930, 932, 934, and 936. Additionally, some nodes may have unknown data due to a lack of patient information or lack of exam/test results, such as node 906.

In contrast to the recommendation model, during training, Bayesian network 900 learns conditional probabilities of each node 901 being selected based on historical patient data. The conditional probabilities are used after training, during an inference stage, to predict a treatment most likely to be applied to a patient based on the historical patient data, which in some cases may be different from a treatment recommended by the recommendation model.

For example, node 906 may represent a current state of the patient, based on prior clinico-pathological variables represented by nodes 902 and 904. At node 906, two treatment options are recommended by the recommendation model: a first treatment option corresponding to node 908, and a second treatment option corresponding to node 910. The recommendation model may provide criteria for selecting the first treatment option vs. the second treatment option. The patient data corresponding to the criteria may not be available, whereby the recommendation model may not be able to provide a clear treatment recommendation. The recommendation model may select one of the first treatment option and the second treatment option.

However, Bayesian network 900 may learn, from the historical patient data, that the second treatment option at node 910 has an 80% chance of being selected by care providers, while the first treatment option at node 908 has a 20% chance of being selected by care providers. As a result, Bayesian network 900 may predict the selection of the second treatment option as opposed to the first treatment option. As described in greater detail below, the second treatment option predicted by Bayesian network 900 may be compared with the selected treatment option recommended by the recommendation system, for verification of the selected treatment option. If the selected treatment option outputted by the recommendation model matches the predicted treatment option outputted by the prediction model, the clinical recommendation system may display a response of the LLM to the care provider, as described above in reference to FIG. 4.

Returning to method 500, at 506, method 500 includes generating training pairs comprising natural language prompts and ground truth responses, based on the trained recommendation model. In various embodiment, the natural language prompts and the ground truth responses may be generated manually by human experts. For example, human AI trainers may provide conversations in which the AI trainers play both sides of the conversation, meaning the user and a theoretical AI assistant (e.g., the clinical recommendation system). The AI trainers may use the recommendation model to generate the conversations. For example, an AI trainer may generate a natural language question based on a set of patient data corresponding to a decision point of the recommendation model, and the AI trainer may generate a natural language response that may be outputted by the AI assistant based on a recommended treatment corresponding to the decision point. The natural language question and the natural language response may be included in a training pair of the training data, with the natural language response acting as ground truth data. In this way, a plurality of natural language training pairs may be generated by the human experts for training an LLM to recommend a treatment for a patient based on a natural language query posed by a care provider.

Each training pair may include a natural language prompt as input data, and a corresponding natural language response generated by an AI trainer as ground truth data. For example, one natural language prompt may be “Given stage 4 lung cancer with TNM status [xxx] and surgical margin [yyy], what should be the next steps?”, and the corresponding natural language response may be “Given stage 4 lung cancer with TNM status [xxx] and surgical margin [yyy], chemotherapy is recommended”.

In some embodiments, natural language training pairs may be generated by one or more chat-bots that traverse the recommendation model and generate plausible questions based on data at various decision points. The chat-bot-generated training pairs may be used in conjunction with the human-generated training pairs. For example, the chat-bot-generated training pairs may be merged with the human-generated training pairs during training. Alternatively, the training may be performed on different training data in stages. For example, the human-generated training pairs may be used to train the LLM in a first stage, and the chat-bot-generated training pairs may be used to train the LLM in a second stage, or the chat-bot-generated training pairs may be used to train the LLM in the first stage, and the human-generated training pairs may be used to train the LLM in a second stage.

At 508, the method includes training the LLM on a general-domain NLP data set. The general-domain NLP data set may be a publicly available data set associated with the LLM that may be used to train the LLM to respond to general questions in an intelligent manner. A supervised learning framework will be used for such re-tuning. However, the general-domain NLP data set may not include training data specific to the clinical guidelines.

At 510, method 500 includes fine-tuning the LLM after training is finished with the general-domain NLP data set, by training the LLM on the generated natural language training pairs based on the clinical guidelines (e.g., either or both of the human-generated training pairs and the chat-bot-generated training pairs). The LLM may be trained on the generated natural language pairs using supervised learning. By fine-tuning the LLM on the generated natural language pairs, an accuracy of the LLM in responding to requests for clinical guidance based on patient data may be increased.

At 512, method 500 includes collecting a plurality of sample outputs of the fine-tuned LLM for a given set of input patient data, and ranking each output of the plurality of sample outputs using human experts. In other words, a number of the natural language prompts generated by the human experts and/or chat-bots for training the LLM may be randomly selected from the training set. For each of the selected prompts, several alternative outputs of the fine-tuned LLM may be sampled. The AI trainers may then rank the alternative outputs. In various embodiments, the outputs may be ranked based on a proximity of an embedding of the outputs in a feature space, as measured by Mahalanobis, Wasserstein, Euclidean and/or similar types of distance measures.

At 514, method 500 includes training a reward model on the ranked outputs, using supervised learning. The reward model may be a neural network model, such as a CNN. Under the reinforcement learning framework, models that generate outputs with closer proximity to human experts would be rewarded.

At 516, method 500 includes optimizing the trained and fine-tuned LLM (e.g., trained LLM 310 of FIG. 3A) using reinforcement learning. Optimizing the fine-tuned LLM may increase an accuracy and/or performance of the LLM when answering queries about treatment alternatives for patients based on current and prior clinico-pathological variables provided by a care provider. As opposed to supervised learning, where a model is provided with examples of desired outputs during training, reinforcement learning trains the model to generate outputs that maximize a reward. An advantage of reinforcement learning is that it does not rely on labeled training data, whereby larger and more comprehensive datasets may be used for training. For example, during the optimization training process, large corpora of data may be used, including petabytes of web-crawler output, Internet-based books, data extracted from online encyclopedias and similar reference materials, and the like. The corpora of data may include general information, and information related to health care, biology, medicine, patient treatments etc.

When trained using reinforcement learning, the model may learn a set of policies to apply to information provided to the model to output optimal responses, including information that the model has not been trained on. In general, optimizing the fine-tuned LLM using reinforcement learning includes retraining the fine-tuned LLM using a reinforcement algorithm and the reward model trained at 514. The optimization of the fine-tuned LLM is described in greater detail below in reference to FIG. 6.

At 518, method 500 includes deploying the optimized LLM for use in the clinical recommendation system. The application of the optimized LLM by the clinical recommendation system to respond to queries posed by care providers is described in greater detail below in reference to FIG. 7.

At 520, method 500 includes collecting and storing new patient data for further training of the prediction model. Patient data including outcome data may be periodically collected from an EMR of the health care system to be incorporated into training data (e.g., outcome probability data 209 of FIG. 2) for ongoing training of prediction model. For example, a care provider may submit a prompt to the clinical recommendation system requesting guidance regarding a treatment for a patient. The care provider may include in the prompt current and prior patient data on which the treatment decision should be based. The clinical recommendation system may provide a response to the prompt indicating a recommended treatment for the patient. The care provider may treat the patient in accordance with the recommended treatment. An outcome of the recommended treatment may be observed at a later date, and may be recorded in the EMR at the later date. At a time after the later date, the clinical recommendation system may retrieve the outcome data recorded in the EMR. For example, the clinical recommendation system may periodically retrieve batches of updated patient data including updated outcome data. The updated patient data including the outcome of the recommended treatment may be used to train the prediction model. After the prediction model has been trained on the updated patient data, the prediction model may more accurately predict a probability of the recommended treatment being applied to other patients with similar patient data as the patient.

The new patient data may also be stored for periodically retraining the LLM and/or the recommendation model. For example, a new training data set for retraining the LLM may be periodically created by the human experts in the manner described above, which may incorporate the new patient data including the outcome data. Additionally, the recommendation model may be periodically retrained as the clinical guidelines used for training the recommendation model are changed over time, in response to the new patient data.

Referring now to FIG. 6, an exemplary method 600 is shown for optimizing an LLM such as LLM 120 of FIG. 1 and/or trained LLM 310 of FIG. 3A using reinforcement learning. Method 600 may be performed by a processor of a clinical recommendation system, such as processor 104 of clinical recommendation system 102 of FIG. 1, based on instructions stored in a non-transitory memory of the clinical recommendation system, such as in AI model module 108 and/or training module 110.

As described above, reinforcement learning trains the LLM to generate outputs that maximize a reward, where the reward is determined by the trained reward model. During the optimization process, the LLM may be trained on a training dataset including natural language prompts. The natural language prompts may be generated manually by human experts, as described above in reference to method 500 of FIG. 5. However, the training dataset may not include responses generated by the human experts (e.g., ground truth data) used for supervised learning. During each iteration of training, a natural language prompt may be submitted to the LLM, and a reward may be allocated to an output of the LLM by the reward model. Parameters of the LLM may then be adjusted based on the allocated reward in accordance with a reinforcement training algorithm. In various embodiments, the reinforcement training algorithm may be the proximal policy optimization (PPO) reinforcement algorithm commonly used on LLMs.

Method 600 starts at 602, where the method includes initializing the PPO model based on the trained LLM. During PPO initializing, smaller updates may be allowed to the policy that stabilize model training.

At 604, method 600 includes selecting a prompt from the training data set, and at 606, the prompt is submitted to the trained LLM.

At 608, method 600 includes calculating a reward for a response to the prompt outputted by the trained LLM. The reward may be a numerical score assigned to the prompt by the reward model. The prompt and the response may be inputted into the reward model, and the reward model may output the reward based on human-ranked training data used to train the reward model, as described above.

At 610, method 600 includes updating a plurality of parameters of the trained LLM based on the allocated reward in accordance with the PPO model. PPO restricts large policy updates, thereby stabilizing the training process. A reward or a punishment does not allow a large change in policy, and the policy is restricted by the clip a ratio that is probability of taking action now divided by the previous one.

At 612, method 600 includes determining whether the training process has stabilized and/or converged. A convergence may be achieved when a performance of the LLM indicates that the rewards assigned to responses are consistently maximized, and the performance is no longer improving. For example, a first performance of the LLM over a first, shorter number of training iterations may be compared with a second performance of the LLM over a second, longer number of training iterations. If the first performance is within a threshold difference of the second performance, it may be inferred that the performance is no longer improving.

If at 612 the training process has not stabilized and/or converged, method 600 proceeds back to 604, and training is continued with a new prompt selected from the training data. Alternatively, if at 612 the training process has stabilized and/or converged, method 600 proceeds to 610. At 610, the optimization training is stopped, and the optimized LLM (e.g., optimized LLM 360 of FIG. 3B) is deployed to be used by the clinical recommendation system. Method 600 ends.

FIG. 7 shows an exemplary method 700 for providing clinical guidance to care providers via a clinical recommendation system, where the clinical recommendation system relies on an optimized LLM to process queries submitted by the care providers. The optimized LLM may be a non-limiting example of optimized LLM 360 of FIG. 3B. Method 700 may be performed by a processor of the clinical recommendation system, such as processor 104 of clinical recommendation system 102 of FIG. 1, based on instructions stored in a non-transitory memory of the clinical recommendation system, such as in inference module 111. Method 700 may be performed in real time, where no intentional delay is introduced when generating a response to a query submitted to the clinical recommendation system by a care provider.

Method 700 begins at 702, where method 700 includes receiving a query from a care provider. The query may be a question posed by the care provider regarding a treatment for a patient being treated by the care provider. For example, the care provider may not be sure of a possible treatment, or may have two or more options for treating the patient, and may wish to consult with the clinical recommendation system to determine which of the two or more options to select. The care provider may submit the query via a UI of the clinical recommendation system, such as UI 404 of FIG. 4. In some embodiments, the care provider may submit the query in written form, for example via a keyboard. In other embodiments, the care provider may submit a query by voice by speaking into a microphone. The query may be posed in natural language, for example, as a question or instruction. As an example, a query submitted by a care provider may be, “Given stage 4 lung cancer with TNM status [xxx] and surgical margin [yyy], what should be the next steps?”.

At 704, method 700 includes translating the query into a prompt to be submitted to the LLM. Translating the query into the prompt may include converting an audio clip or audio file received by the clinical recommendation system via a microphone into a written form. Additionally, a format of the query may be adjusted to an input format of the LLM. For example, the query may be posed by the care provider as a question, and the question may be converted into an instruction for the LLM.

At 706, method 700 includes submitting the prompt to the LLM. The prompt may be inputted into the LLM, and the LLM may output a response to the prompt. The response may include clinically explainable information, such as why the patient is at a certain node or stage in the recommendation model (and guidelines). The response may further indicate advised or suggested next steps in accordance with clinical guidelines, based on the patient's position and existing clinical/pathological variables.

At 708, method 700 includes extracting patient data included in the submitted prompt. In various embodiments, the patient data may be extracted using a tokenizer (e.g., tokenizer 410 of FIG. 4).

At 710, method 700 includes predicting a most probable treatment based on the extracted patient data, using a prediction model (e.g., prediction model 412). As described above, the prediction model may take as input current and prior clinico-pathological variables of a patient (e.g., the extracted patient data), and output a treatment alternative with a highest probability of being selected for the patient, based on population statistics and historical patient data.

At 712, method 700 includes extracting the treatment recommended by the LLM from the response outputted by the LLM, with the aid of the tokenizer, in a manner similar to the extraction of the patient data.

At 714, method 700 includes determining whether the extracted, recommended treatment by the LLM matches the predicted treatment outputted by the prediction model. The outputs may be compared based on a proximity of their embedding in a feature space, as measured by Mahalanobis, Wasserstein, Euclidean and/or similar kind of distance measures, and top-ranking recommendations would be provided. Further, the recommendations may be matched to a decision tree of clinical guidelines for sanity checks.

If the recommended treatment matches the predicted treatment, method 700 proceeds to 716. At 716, method 700 includes delivering the response outputted by the LLM to the care provider. In various embodiments, the response may be displayed to the care provider on a display device via a UI of the clinical recommendation system (e.g., via UI 404 and/or display device 134). In some embodiments, the response may be converted into an audio signal using text-to-voice conversion software, and the audio signal may be delivered to the care provider via a speaker or device including a speaker coupled to the clinical recommendation system. For example, the care provider may not have immediate access to a display device, and the care provider may select to have the response routed to a smart phone of the care provider.

At 718, method 700 includes saving the recommended treatment to a patient decision workflow of the patient.

Alternatively, if at 714 the recommended treatment does not match the predicted treatment, method 700 proceeds to 720. At 720, method 700 includes logging an inconsistency between the recommended treatment and a current or historical practice, based on the population statistics and historical patient data. The inconsistency may indicate that the clinical guidelines on which the recommendation model is based is not aligned with current practice. In such cases, an intervention may be performed to ensure that the current practice adheres to the clinical guidelines. Alternatively, if the current practice is shown to lead to a higher probability of positive outcomes, the clinical guidelines may be updated. In this way, the clinical guidelines may be maintained up to date, and alignment with the clinical guidelines may be facilitated.

Thus, the proposed clinical recommendation system may support care providers by providing clinical guidance with respect to patient treatment in a more natural way than the manner in which clinicians currently interact with clinical guidelines. Rather than having to search and navigate through digital and/or online clinical guidelines using a GUI, which may entail a sequence of actions including reading, selecting control elements of the GUI, typing information into the GUI, and waiting for results, the guidance may be requested in natural language, for example, via a microphone. By providing the care providers with the clinical guidance in a more efficient and faster manner, use of the clinical recommendation system and the clinical guidelines on which it is based may be ensured, encouraged, and increased, leading to a higher degree of positive outcomes.

The clinical recommendation system may provide a clinically explainable disease state of a patient and recommend a next course of action (e.g., a treatment) based on clinical guidelines, without burdening clinicians with time-consuming and/or cumbersome interactions with a computerized system. As a result of the natural language-based interaction, the clinical recommendation system would reduce the digital device interactions of the care providers, freeing the care providers to devote more time with patients. An additional advantage of the proposed clinical recommendation system is that care providers and patients may be provided a greater degree of transparency with respect to how clinical decisions are made and on what data the clinical decisions are based. As a result, clinical recommendation system may facilitate a knowledgeable decision-making process for the patient and encourage a greater degree of trust. Further, the proposed clinical recommendation system may reduce variability in the clinical decision-making process, and introduce a checklist for the clinician based on prior population statistics that is tractable and could allow for more efficient reimbursement.

The technical effect of providing clinical recommendations to care providers via a natural language interface is that the care providers may request and receive guidance based on clinical guidelines in a more efficient and faster manner, leading to increased reliance on the clinical guidelines, increased positive patient outcomes, and more clinician time spent with patients.

The disclosure also provides support for a method for a clinical recommendation system, the method comprising: generating an automated response to a natural language query received from a care provider in real time using a first, large language model (LLM) of the clinical recommendation system, the automated response including a recommended treatment of a patient, the LLM trained on a first set of training data generated by applying a second, recommendation model to anonymized patient data, the recommendation model based on clinical guidelines, verifying the automated response outputted by the LLM using a third, prediction model, and in response to the automated response being verified, storing the automated response in a patient decision workflow of the patient, the patient decision workflow including a record of clinical recommendations made with respect to the patient using the clinical recommendation system, and displaying the automated response on a display device of the clinical recommendation system. In a first example of the method, the recommendation model includes one of a decision tree and a knowledge graph. In a second example of the method, optionally including the first example, the prediction model includes one of a Bayesian network and a convolutional neural network (CNN). In a third example of the method, optionally including one or both of the first and second examples, the prediction model is trained on a second set of training data generated by applying the recommendation model to the anonymized patient data. In a fourth example of the method, optionally including one or more or each of the first through third examples, verifying the automated response outputted by the LLM using the prediction model further comprises: extracting the patient data from the natural language query, extracting the recommended treatment from the automated response outputted by the LLM, determining a second treatment with a highest probability of being selected based on the extracted patient data, using the prediction model, in response to the recommended treatment matching the second treatment, verifying the automated response, and in response to the recommended treatment not matching the second treatment, reporting an inconsistency between the recommended treatment and the clinical guidelines, the clinical guidelines including the National Comprehensive Cancer Network (NCCN) Clinical Practice Guidelines in Oncology (NCCN Guidelines®). In a fifth example of the method, optionally including one or more or each of the first through fourth examples, the recommended treatment is based on a previous recommended treatment stored in a patient decision workflow of a previous patient. In a sixth example of the method, optionally including one or more or each of the first through fifth examples, verifying the automated response outputted by the LLM further comprises comparing a recommended treatment of the automated response with a previous recommended treatment of a previous patient stored in a patient decision workflow of the previous patient. In a seventh example of the method, optionally including one or more or each of the first through sixth examples, the method further comprises: receiving patient outcome data corresponding to the patient from an electronic medical record (EMR), and using the patient outcome data to further train the prediction model. In a eighth example of the method, optionally including one or more or each of the first through seventh examples, training the LLM on the first set of training data further comprises: training the LLM on a general-domain NLP dataset, fine-tuning the LLM by training the LLM on natural language training pairs using supervised learning, the natural language training pairs including a prompt and a ground truth response to the prompt, and optimizing the LLM by training the LLM on various corpora of unlabeled data, using reinforcement learning. In a ninth example of the method, optionally including one or more or each of the first through eighth examples, the natural language training pairs are generated by at least one of human experts using the recommendation model as a guide, and one or more chat-bots that traverse the recommendation model. In a tenth example of the method, optionally including one or more or each of the first through ninth examples, fine-tuning the LLM by training the LLM on natural language training pairs using supervised learning further comprises training the LLM on a first set of training pairs generated manually by the human experts, and then training the LLM on a second set of training pairs generated by the one or more chat-bots. In a eleventh example of the method, optionally including one or more or each of the first through tenth examples, optimizing the LLM by training the LLM on various corpora of unlabeled data using reinforcement learning further comprises: inputting a set of generated natural language training prompts into the fine-tuned LLM a plurality of times to generate a plurality of alternative outputs, receiving a ranking of the plurality of alternative outputs by the human experts, training a reward model on the generated natural language training prompts and the alternative outputs using the rankings as ground truth data, and training the LLM using reinforcement learning based on the reward model and a reinforcement learning algorithm. In a twelfth example of the method, optionally including one or more or each of the first through eleventh examples, the natural language query is received from the care provider by voice via a microphone, and/or the response is delivered to the care provider via an audio signal delivered over a speaker.

The disclosure also provides support for a clinical recommendation system, comprising a processor and a non-transitory memory storing instructions that when executed, cause the processor to: receive a natural language query from a care provider via a user interface of the clinical recommendation system, the natural language query including patient data of a patient, generate a response to the natural language query using a large language model (LLM), the response including a recommended treatment based on the patient data, store the response in a patient decision workflow of the patient, and deliver the response to the care provider via a display device and/or a speaker of the clinical recommendation system, wherein the LLM is trained using supervised learning on human- or chat-bot-generated natural language training pairs generated in accordance with a recommendation model based on clinical guidelines, and optimized by training the LLM on various corpora of unlabeled data using reinforcement learning based on a reward model trained on human-ranked training data. In a first example of the system, the recommendation model is one of a decision tree and a knowledge graph including decision points where different treatments are recommended based on the patient data. In a second example of the system, optionally including the first example, further instructions are stored in the non-transitory memory that, when executed, cause the processor to: verify the response using a prediction model trained to take the patient data as input, and output a treatment that has a highest probability of being selected based on population statistics and/or historical patient data, using the recommendation model as a guide. In a third example of the system, optionally including one or both of the first and second examples, verifying the response using the prediction model comprises: extracting the patient data from the natural language query, extracting a first treatment from the response outputted by the LLM, determining a second treatment with the highest probability of being selected based on the extracted patient data, using the prediction model, in response to the first treatment matching the second treatment, verifying the response, and in response to the first treatment matching the second treatment, reporting an inconsistency between the recommended treatment and the clinical guidelines.

The disclosure also provides support for a method for providing clinical guidance to care providers via a clinical recommendation system, the method comprising: receiving a query from a care provider, the query including data of a patient, translating the query into a natural language prompt, submitting the natural language prompt to a large language model (LLM), the LLM trained to output a response with a recommended treatment for the patient based on the patient data and one or more sets of digital clinical guidelines, extracting the patient data from the natural language prompt, predicting a most probable treatment for the patient based on the extracted patient data, using a prediction model trained on population statistics and anonymized historical patient data, extracting the recommended treatment included from the response generated by the LLM, comparing the recommended treatment with the predicted most probable treatment, in response to the recommended treatment matching the predicted most probable treatment, storing the recommended treatment in a patient decision workflow of the patient and delivering the response to the care provider via a display device and/or a speaker of the clinical recommendation system, and in response to the recommended treatment not matching the predicted most probable treatment, logging an inconsistency of the recommended treatment with a current practice. In a first example of the method, the training of the LLM includes: fine-tuning the LLM by training the LLM on natural language training pairs using supervised learning, each natural language training pair including a prompt and a ground truth response to the prompt generated by at least one of a human expert and a chat-bot using a recommendation model trained on the one or more sets of digital clinical guidelines as a guide, and optimizing the LLM by training the LLM on various corpora of unlabeled data, using reinforcement learning, based on a reward model trained on human-ranked responses to a selected set of generated prompts. In a second example of the method, optionally including the first example, the recommendation model includes a decision tree based on a set of digital clinical guidelines, the recommendation model taking patient data as input, traversing the decision tree in accordance with the patient data, and outputting a recommended treatment at a decision point of the decision trec.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative and should not be construed to be limiting in any manner.

1. A method for a clinical recommendation system, the method comprising:
generating an automated response to a natural language query received from a care provider in real time using a first, large language model (LLM) of the clinical recommendation system, the automated response including a recommended treatment of a patient, the LLM trained on a first set of training data generated by applying a second, recommendation model to anonymized patient data, the recommendation model based on clinical guidelines;
verifying the automated response outputted by the LLM using a third, prediction model; and
in response to the automated response being verified, storing the automated response in a patient decision workflow of the patient, the patient decision workflow including a record of clinical recommendations made with respect to the patient using the clinical recommendation system, and displaying the automated response on a display device of the clinical recommendation system.

2. The method of claim 1, wherein the recommendation model includes one of a decision tree and a knowledge graph.

3. The method of claim 1, wherein the prediction model includes one of a Bayesian network and a convolutional neural network (CNN).

4. The method of claim 1, wherein the prediction model is trained on a second set of training data generated by applying the recommendation model to the anonymized patient data.

5. The method of claim 1, wherein verifying the automated response outputted by the LLM using the prediction model further comprises:
extracting the patient data from the natural language query;
extracting the recommended treatment from the automated response outputted by the LLM;
determining a second treatment with a highest probability of being selected based on the extracted patient data, using the prediction model;
in response to the recommended treatment matching the second treatment, verifying the automated response; and
in response to the recommended treatment not matching the second treatment, reporting an inconsistency between the recommended treatment and the clinical guidelines, the clinical guidelines including the National Comprehensive Cancer Network (NCCN) Clinical Practice Guidelines in Oncology (NCCN Guidelines®) or equivalent local guidelines.

6. The method of claim 1, wherein the recommended treatment is based on a previous recommended treatment stored in a patient decision workflow of a previous patient.

7. The method of claim 1, wherein verifying the automated response outputted by the LLM further comprises comparing a recommended treatment of the automated response with a previous recommended treatment of a previous patient stored in a patient decision workflow of the previous patient.

8. The method of claim 1, further comprising:
receiving patient outcome data corresponding to the patient from an electronic medical record (EMR); and
using the patient outcome data to further train the prediction model.

9. The method of claim 1, wherein training the LLM on the first set of training data further comprises:
training the LLM on a general-domain NLP dataset;
fine-tuning the LLM by training the LLM on natural language training pairs using supervised learning, the natural language training pairs including a prompt and a ground truth response to the prompt; and
optimizing the LLM by training the LLM on various corpora of unlabeled data, using reinforcement learning.

10. The method of claim 9, wherein the natural language training pairs are generated by at least one of human experts using the recommendation model as a guide, and one or more chat-bots that traverse the recommendation model.

11. The method of claim 10, wherein fine-tuning the LLM by training the LLM on natural language training pairs using supervised learning further comprises training the LLM on a first set of training pairs generated manually by the human experts, and then training the LLM on a second set of training pairs generated by the one or more chat-bots.

12. The method of claim 10, wherein optimizing the LLM by training the LLM on various corpora of unlabeled data using reinforcement learning further comprises:
inputting a set of generated natural language training prompts into the fine-tuned LLM a plurality of times to generate a plurality of alternative outputs;
receiving a ranking of the plurality of alternative outputs by the human experts;
training a reward model on the generated natural language training prompts and the alternative outputs using the rankings as ground truth data; and
training the LLM using reinforcement learning based on the reward model and a reinforcement learning algorithm.

13. The method of claim 1, wherein the natural language query is received from the care provider by voice via a microphone, and/or the response is delivered to the care provider via an audio signal delivered over a speaker.

14. A clinical recommendation system, comprising a processor and a non-transitory memory storing instructions that when executed, cause the processor to:
receive a natural language query from a care provider via a user interface of the clinical recommendation system, the natural language query including patient data of a patient;
generate a response to the natural language query using a large language model (LLM), the response including a recommended treatment based on the patient data;
store the response in a patient decision workflow of the patient; and
deliver the response to the care provider via a display device and/or a speaker of the clinical recommendation system; wherein
the LLM is trained using supervised learning on human- or chat-bot-generated natural language training pairs generated in accordance with a recommendation model based on clinical guidelines, and optimized by training the LLM on various corpora of unlabeled data using reinforcement learning based on a reward model trained on human-ranked training data.

15. The clinical recommendation system of claim 14, wherein the recommendation model is one of a decision tree and a knowledge graph including decision points where different treatments are recommended based on the patient data.

16. The clinical recommendation system of claim 14, wherein further instructions are stored in the non-transitory memory that, when executed, cause the processor to:
verify the response using a prediction model trained to take the patient data as input, and output a treatment that has a highest probability of being selected based on population statistics and/or historical patient data, using the recommendation model as a guide.

17. The clinical recommendation system of claim 16, wherein verifying the response using the prediction model comprises:
extracting the patient data from the natural language query;
extracting a first treatment from the response outputted by the LLM;
determining a second treatment with the highest probability of being selected based on the extracted patient data, using the prediction model;
in response to the first treatment matching the second treatment, verifying the response; and
in response to the first treatment matching the second treatment, reporting an inconsistency between the recommended treatment and the clinical guidelines.

18. A method for providing clinical guidance to care providers via a clinical recommendation system, the method comprising:
receiving a query from a care provider, the query including data of a patient;
translating the query into a natural language prompt;
submitting the natural language prompt to a large language model (LLM), the LLM trained to output a response with a recommended treatment for the patient based on the patient data and one or more sets of digital clinical guidelines;
extracting the patient data from the natural language prompt;
predicting a most probable treatment for the patient based on the extracted patient data, using a prediction model trained on population statistics and anonymized historical patient data and/or prior recommended treatments;
extracting the recommended treatment included from the response generated by the LLM;
comparing the recommended treatment with the predicted most probable treatment;
in response to the recommended treatment matching the predicted most probable treatment, storing the recommended treatment in a patient decision workflow of the patient and delivering the response to the care provider via a display device and/or a speaker of the clinical recommendation system; and
in response to the recommended treatment not matching the predicted most probable treatment, logging an inconsistency of the recommended treatment with a current practice.

19. The method of claim 18, wherein the training of the LLM includes:
fine-tuning the LLM by training the LLM on natural language training pairs using supervised learning, each natural language training pair including a prompt and a ground truth response to the prompt generated by at least one of a human expert and a chat-bot using a recommendation model trained on the one or more sets of digital clinical guidelines as a guide; and
optimizing the LLM by training the LLM on various corpora of unlabeled data, using reinforcement learning, based on a reward model trained on human-ranked responses to a selected set of generated prompts.

20. The method of claim 19, wherein the recommendation model includes a decision tree based on a set of digital clinical guidelines, the recommendation model taking patient data as input, traversing the decision tree in accordance with the patient data, and outputting a recommended treatment at a decision point of the decision tree.
